(19) United States
(12) Patent Application Publication
(10) Pub. No.: US 2011/0070206 A1
(43) Pub. Date: Mar. 24, 2011

(54) METHODS OF APPLYING PHYSICAL STIMULI TO CELLS

(75) Inventors: Clinton Rubin, Port Jefferson, NY (US); Stefan Judex, Port Jefferson, NY (US); Yen Kim Luu, Albuquerque, NM (US)

(73) Assignee: The Research Foundation of State University Of New York, Albany, NY (US)

(21) Appl. No.: 12/919,533

(22) PCT Filed: Mar. 2, 2009
(86) PCT No.: PCT/US09/35777

§ 371 Date: Dec. 1, 2010

Related U.S. Application Data
(60) Provisional application No. 61/032,942, filed on Feb. 29, 2008.

(51) Int. Cl. C12N 5/07; C12N 5/10; C12Q 1/02; A61K 35/12; A61H 1/00

(52) U.S. Cl. 424/93.7; 435/375; 435/29; 601/1

(57) ABSTRACT

We describe herein methods for applying physical stimuli to cells, including mesenchymal stem cells, in culture or in vivo. These methods can be applied in, and are expected to benefit subjects in, a great variety of circumstances that arise in the context of, for example, traumatic injury (including that induced by surgical procedures), wound healing (of the skin and other tissues), cancer therapies (e.g., chemotherapy or radiation therapy), tissue transplantation (e.g., bone marrow transplantation), and aging. More generally, the present methods apply where patients would benefit from an increase in the number of cells (e.g., stem cells) within a given tissue and, ex vivo, where it is desirable to increase the proliferation of cells (e.g., stem cells) for scientific study, inclusion in devices bearing cells (e.g., polymer or hydrogel-based implants), and administration to patients.

### CROSS REFERENCE TO RELATED APPLICATIONS

This application claims priority to U.S. Application Ser. No. 61/032,942, filed on Feb. 29, 2008. For the purpose of any U.S. patent that may issue based on the present application, U.S. Application Ser. No. 61/032,942 is hereby incorporated by reference in its entirety.

### FEDERALLY SPONSORED RESEARCH OR DEVELOPMENT

The work described below was support by Grant No. AR 43498 which was awarded by the National Institutes of Health. The Government has certain rights in the invention.

### TECHNICAL FIELD

This invention relates to methods for altering the differentiation and proliferation of cells, including stem cells, in cell culture or in patients who have had, for example, a traumatic injury. The methods can also be used, for example, to counteract a side effect of chemotherapy or radiation therapy or to improve the outcome of a transplant, such as a bone marrow transplant.

### SUMMARY

The present invention is based, in part, on our discovery that applying reasonably brief periods of low-magnitude, high-frequency mechanical signals to a cell (or population of cells, whether homogeneous or heterogeneous and whether found in cell culture, tissue culture, or within a living organism (e.g., a human)) on a periodic basis (e.g., a daily basis) can increase cellular proliferation and/or influence cell fate (i.e., influence one or more of the characteristics of a cell or alter the type of cell a precursor cell would have otherwise become).

The methods can be used to produce populations of cells, or to more quickly produce populations of cells, that can be used in various manufacturing processes. For example, the cells subjected to LMMS can be yeast cells used in any otherwise conventional process in the brewing industry. In other instances, the cells can be prokaryotic or eukaryotic cells used to produce therapeutic proteins (e.g., antibodies, other target-specific molecules such as aptamers, blood proteins, hormones, or enzymes). In other instances, the cells can be generated in cell or tissue culture for use in tissue engineering (e.g., by way of inclusion in a device, such as a scaffold, mesh, or gel (e.g., a hydrogel)).

Where the stimulus is applied in vivo, it may be applied to an organism from which tissue will be harvested (for, for example, use in a tissue engineering construct or for transplantation to a recipient). Alternatively, or in addition, the stimulus can be applied to a patient as a therapeutic treatment. The patient may have, for example, a damaged or defective organ or tissue. The damage or defect can be one that results from any type of trauma or it may be associated with nutritional deficiencies (e.g., a high fat diet). More generally, the patient can be any subject who would benefit from an increase in the number of stem cells within their tissues (e.g., an adult or elderly patient) or from an increase in the number of stem cells that differentiate into non-adipose cells. The signal can be applied to the patient by virtue of a platform on which the patient stands or lies. Alternatively, the signal can be applied more locally to a region or tissue of interest (e.g., by a handheld device).

The damaged or defective organs or tissues can include those affected by a wide range of medical conditions including, for example, traumatic injury (including injury induced in the course of a surgical or other medical procedure, such as an oncologic resection or chemotherapy), tissue damaging diseases, neurodegenerative diseases (e.g., Parkinson's Disease or Huntington's Disease), demyelinating diseases, congenital malformations (e.g., hypospadias), limb malformations, neural tube defects, and tissue loss, malfunction, or malformation resulting from or associated with an infection, compromised diet, or environmental insult (e.g., pollution or exposure to a damaging substance such as radiation, tar, nicotine, or alcohol). For example, the patient can have cardiac valve damage, tissue wasting, tissue inflammation, tissue scarring, ulcers, or undesirably high levels of adipose tissue (e.g., within the liver).

Accordingly, the invention features methods of increasing the proliferation and/or differentiation of a cell within the body of an organism (i.e., in vivo), a cell that has been removed from an organism and placed in culture, or a single-celled organism (e.g., a fungal or bacterial cell). A variety of cell types of diverse histological origins are amenable to the present methods. The cell can be a cell that has been removed from an organism and placed in culture for either a brief period (e.g., as a tissue explant) or for an extended length of time (e.g., an established cell line). The cell can be any type of stem cell, for example an embryonic stem cell or an adult stem cell. Adult stem cells can be harvested from many types of adult tissues, including bone marrow, blood, skin, gastrointestinal tract, dental pulp, the retina of the eye, skeletal muscle, liver, pancreas, and brain. The methods are not limited to undifferentiated stem cells and can include those cells that have committed to a partially differentiated state. More specifically, the cell can be a mesenchymal stem cell, a hematopoietic stem cell, an endothelial stem cell, or a neuronal stem cell. Such a partially differentiated cell may be a precursor to an adipocyte, an osteocyte, a hepatocyte, a chondrocyte, a neuron, a glial cell, a myocyte, a blood cell, an endothelial cell, an epithelial cell, a fibroblast, or a endocrine cell. Established cell lines, for example, embryonic stem cell lines, are also embraced by the methods, as are bacterial cells, including E. coli and other bacteria that can be used to produce recombinant proteins, and yeast (e.g., yeast suitable for brewing beer or other alcoholic beverages). Optionally, the cell can be one that naturally expresses a desirable gene product or that has been modified to express one or more exogenous genes. The methods can be applied to cells of mammalian origin (e.g., humans, mice, rats, canines, cows, horses, felines, and ovines) as well as cells from non-mammalian sources (e.g., fish and birds).

Regardless of the cell type that is used, the methods can be carried out by providing to the cell, or a subject in which the cell is found, a low-magnitude, high-frequency physical signal. The physical signal is preferably mechanical, but can also be another non-invasive modality (e.g., a signal generated by acceleration, electric fields, or transcutaneous ultrasound). The signal can be supplied on a periodic basis and for a time sufficient to achieve a desirable outcome (e.g., one or more of the outcomes described herein). For example, the signal can be supplied to increase or enhance the proliferation rate of a cell in culture. For example, a cell or a population of cells, whether homogenous or heterogeneous, may divide or double faster (e.g., 1-500% faster) than a comparable cell or population of cells, under the same or essentially similar circumstances, that has not been exposed to the present mechanical signals.

The signal can also be supplied to a whole organism to increase the proliferation rate of particular target cell populations. Because our data indicate these physical signals can influence the fate of mesenchymal stem cells, the present methods can also be used to help retain or restore any tissue type, with the likely exception of adipose tissue. For example, the present methods can be used to promote bone marrow viability and to direct the proliferation and controlled differentiation of stem cells, including those placed in cell culture, down specific pathways (e.g., toward differentiated bone cells, liver cells, or muscle cells, rather than toward adipocytes).

The time of exposure to the physical signal can be brief, and the periodic basis on which it is applied may or may not be regular. For example, the signal can be applied almost exactly every so many hours (e.g., once every 4, 8, 12, or 24 hours) or almost exactly every so many days (e.g., at nearly the same time every other day, once a week, or once every 10 or 14 days). Thus, in various embodiments, signals can be applied to a cell daily, but at varied times of the day. Similarly, a cell may miss one or more regularly scheduled applications and resume again at a later point in time. The length of time the signal (e.g., a mechanical signal) is provided can also be highly consistent in each application (e.g., it can be consistently applied for about 2-60 minutes, inclusive (e.g., for about 1, 2, 5, 10, 12, 15, 20, 25 or 30 minutes) or it can vary from one session to the next. Any of the methods can further include a step of identifying a subject (e.g., a human) prior to providing the low-magnitude, high-frequency physical (e.g., mechanical) signal, and the identification process can include an assessment of physical health and the disorder or tissue in need of repair. We may use the terms “subject,” “individual” and “patient” interchangeably. While the present methods are certainly intended for application to human patients, the invention is not so limited. For example, domesticated animals, including cats and dogs, or farm animals can also be treated.

The physical signals can be characterized in terms of magnitude and/or frequency, and are preferably mechanical in nature, induced through the weightbearing skeleton or directly by acceleration in the absence of weightbearing. Useful mechanical signals can be delivered through accelerations of about 0.01-10.0 g, where “g” or “1 g” represents acceleration resulting from the Earth's gravitational field (1.0 g=9.8 m/s/s). Surprisingly, signals of extremely low magnitude, far below those that are most closely associated with strenuous exercise, are effective. These signals can be, for example, of a lesser magnitude than those experienced during walking. Accordingly, the methods described here can be carried out by applying 0.1-1.0 g (e.g., 0.2-0.5 g (e.g., about 0.2 g, 0.3 g, 0.4 g, 0.5 g or signals therebetween (e.g., 0.25 g))). The frequency of the mechanical signal can be about 5-1,000 Hz (e.g., 20-200 Hz (e.g., 30-90 Hz)). For example, the frequency of the mechanical signal can be about 5-100 Hz, inclusive (e.g., about 50-90 Hz (e.g., 50, 60, 70, 80, or 90 Hz) or 20-50 Hz (e.g., about 20, 30, or 40 Hz). A combination of frequencies (e.g., a “chirp” signal from 20-50 Hz), as well as a pulse-burst of physical information (e.g., a 0.5 s burst of 40 Hz, 0.3 g vibration given at least or about every 1 second) can also be used. The magnitudes and frequencies of the acceleration signals that are delivered can be constant throughout the application (e.g., constant during a 10-minute application to a subject) or they may vary, independently, within the parameters set out herein. For example, the methods can be carried out by administering a signal of about 0.2 g and 20 Hz at a first time and a signal of about 0.3 g and 30 Hz at a second time. Further, distinct signals can be used for distinct purposes or aims, such as reversing an undesirable condition or preventing or inhibiting its development.

Any of the present methods can include the step of identifying a suitable source of cells and/or a suitable subject to whom the signal would be administered. Similarly, any of the present methods can be carried out using a human cell.

With respect to particular methods of treatment, the invention encompasses methods of treating a patient by administering to the patient a cell that has been treated, in culture or in a donor prior to harvesting, according to the methods described herein. More specifically, the methods encompass treating a patient who has experienced a traumatic injury to a tissue or who has a tissue damaging disease other than osteopenia or sarcopenia. The method can be carried out by administering to the patient a low magnitude, high frequency mechanical signal on a periodic basis and for a time sufficient to treat the injury or tissue damage. The patient can be, but is not necessarily, a human patient, and the traumatic injury can include a wound to the skin of the patient, such as a cut, burn, puncture, or abrasion of the skin. The traumatic injury can also include a wound to muscle, bone, or an internal organ. Where the injury is caused by disease, the disease can be a neurodegenerative disease.

Other patients amenable to treatment include those undergoing chemotherapy or radiation therapy, or those who have received a bone marrow transplant. Where tissue is transplanted, both the recipient patient and the tissue donor can be treated. The cells may also be treated in culture after harvest but prior to implantation. These methods can be carried out by administering to the patient a low magnitude, high frequency mechanical signal on a periodic basis and for a time sufficient to counteract a harmful side effect of the chemotherapy or radiation therapy on the patient's body or to improve the outcome of the bone marrow transplant. The side effect can be dry or discolored skin, palmar-plantar syndrome, damage to the skin caused by radiation or extravasation of the chemotherapeutic, hair loss, intestinal irritation, mouth sores or ulcers, cell loss from the bone marrow or blood, liver damage, kidney damage, lung damage, or a neuropathy.

The present methods can also be used to slow or reduce a sign or symptom of aging by administering to the patient a low magnitude, high frequency mechanical signal on a periodic basis and for a time sufficient to reduce the depletion of stem cells in the patient (as normally occurs with age). As with other methods described herein, the methods can be carried out on human patients, and elderly patients may be particularly amenable where the natural loss of stem cells occurs.

In another aspect, the invention features methods of preparing a tissue donor. The methods include administering to the donor a low magnitude, high frequency mechanical signal on a periodic basis and for a time sufficient to increase the number of cells in the tissue to be harvested for transplantation. The cells can be stem cells, and the tissue to be harvested can be bone marrow.

The effect of the physical signal on the rate of proliferation for a population of cells in culture can be assessed according to any standard manual or automated method in the art, for example, removing an aliquot of cells from the culture before and after treatment, staining the cells with a vital dye, e.g., trypan blue, and counting the cells in a hemacytometer, tetrazolium salt reagents such as MTT, XTT, MTS, fluorescence activated cell sorting, or Coulter counting. When the treatment is to a whole organism, an aliquot of cells can be removed using biopsy methods.

Where proliferation is enhanced in cell culture, the cells may be associated with a prosthetic or biomaterial. For example, the cells may be associated with a scaffold or substrate suitable for use as a graft, stent, valve, prosthesis, allograft, autograft, or xenograft.

While there are advantages to limiting the present methods to those that require purely physical stimuli, any of the present methods can be carried out in conjunction with other therapies, including those in which drug therapies are used to promote stem cell proliferation.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1A is a dot plot from a flow cytometry analysis of stem cells in general (Sca-1 single positive, upper quadrants), and MSCs specifically (both Sca-1 and Pref-1 positive, upper right quadrant) in the bone marrow of a control mouse.

FIG. 1B is a dot plot from a flow cytometry analysis of stem cells in general (Sca-1 single positive, upper quadrants), and MSCs specifically (both Sca-1 and Pref-1 positive, upper right quadrant) in the bone marrow of a vibrated mouse.

FIG. 1C is a graph comparing the total stem cell number, calculated as % positive cells/total cells for the cell fraction showing highest intensity staining, in a control (CON) to and vibrated (LMMS) mouse.

FIG. 1D is graph comparing the mesenchymal stem cell number, calculated as % positive cells/total cells for the cell fraction showing highest intensity staining, in a control (CON) and vibrated (LMMS) mouse.

FIG. 2A shows distinct cell populations identified in flow cytometry, with stem cells being identified as low forward (FSC) and side (SSC) scatter.

FIG. 2B is a graph showing osteoprogenitor cells, identified as Sca-1(+) cells, residing in the region highlighted as high FSC and SSC, and were 29.9% (p=0.23) more abundant in the bone marrow of LMMS treated animals.

FIG. 2C is a graph showing that the preadipocyte population, identified as Pref-1 (+), Sca-1 (−), demonstrated a trend (+18.5%; p=0.25) towards an increase in LMMS relative to CON animals (C).

FIG. 3A is a graph showing real time RT-PCR analysis of bone marrow samples harvested from untreated (CON) mice and mice subject to 6 weeks LMMS treatment. The osteogenic gene Runx2 was significantly upregulated in the LMMS-treated mice.

FIG. 3B is a graph showing real time RT-PCR analysis of bone marrow samples harvested from untreated (CON) mice and mice subject to 6 weeks LMMS treatment. The adipogenic gene PPARγ was downregulated in the LMMS-treated mice.

FIG. 4A is a graph showing bone volume fraction, as measured in vivo by low resolution μCT, in control (CON) and vibrated (LMMS) mice. LMMS increased bone volume fraction across the entire torso of the animal.

FIG. 4B is a graph showing post-sacrifice, high resolution CT of the proximal tibia in control (CON) and vibrated (LMMS) mice. LMMS significantly increased trabecular bone density.

FIG. 4C is a representative μCT reconstruction at the proximal tibia in a control (CON) mouse.

FIG. 4D is a representative μCT reconstruction at the proximal tibia in a vibrated (LMMS) mouse. Tibiae from LMMS mice showed enhanced morphological properties.

FIG. 5A shows in vivo μCT images used to discriminate visceral and subcutaneous adiposity in the abdominal region of a CON and LMMS mouse. Visceral fat is shown in dark grey, subcutaneous fat in light gray.

FIGS. 5B, 5C, 5D and 5E show linear regressions of calculated visceral adipose tissue (VAT) volume against adipose TG, adipose NEFA, liver TG and liver NEFA, respectively. Linear regressions of calculated visceral adipose tissue (VAT) volume against adipose and liver biochemistry values demonstrated strong positive correlations in CON, and weak correlations in LMMS, as well as generally lower levels for all LMMS biochemical values. N=6 for adipose (FIGS. 5B and 5C), N=10 for liver (FIGS. 5D and 5E). Regressions for adipose TG (p=0.002), adipose NEFA (p=0.03), liver TG (p=0.006) and liver NEFA (p=0.003) were significant for CON animals, but only liver NEFA (p=0.02) was significant for LMMS. Overall, LMMS mice exhibited lower, non-significant correlations in liver TG (p=0.06), adipose TG (p=0.19), and adipose NEFA (p=0.37) to increases in visceral adiposity.

FIG. 6A shows reconstructed in vivo μCT images of total body fat (dark grey) in untreated (CON) and vibrated (LMMS) mice.

FIG. 6B is a graph showing the effect of LMMS treatment on fat volume in two mouse models of obesity. In one, “fat diet”, mice were placed on a high fat diet at the same time that LMMS treatment was initiated. After 12 weeks, mice that received LMMS exhibited 22.2% less fat volume as compared to control mice (CON) that did not receive LMMS treatment. In the other model, “obesity”, mice were maintained on a high fat diet for 3 weeks prior to LMMS treatment. No reduction of fat volume was observed in LMMS mice in the “obesity” model.

FIG. 6C is a graph showing the effect of LMMS treatment on percent adiposity the mouse models shown in FIG. 6B. In the “fat diet” model the percent adiposity, calculated as the relative percentage of fat to total animal volume, LMMS reduced the percent animal adiposity by 13.5% (p=0.017); no effect was observed in the “obesity” model. The lack of a response in the already obese animals suggests that the mechanical signal works primarily at the stem cell development level, as existing fat is not metabolized by LMMS stimulation. Suppression of the obese phenotype was achieved to a degree by stem cells preferentially diverting from an adipogenic lineage.

FIG. 7 is a graph depicting changes in bone density, muscle area and fat area in a group of young osteopenic women subject to LMMS for one year. As measured by CT scans in the lumbar region of the spine, a group of young osteopenic women subject to LMMS for one year (n=24; gray bars±s.e.) increased both bone density (p=0.03 relative to baseline; mg/cm3) and muscle area (p<0.001; cm2), changes which were paralleled by a non-significant increase in visceral fat formation (p=0.22; cm2). Conversely, women in the control group (n=24; white bars±s.e.), while failing to increase either bone density (p=0.93) or muscle area (p=0.52), realized a significant increase in visceral fat formation (p=0.015).

FIG. 8A is a reconstruction of in vivo CT data through longitudinal section of mice showing difference in fat quantity and distribution in CON and LMMS mice. Image represents total body fat in dark gray.

FIG. 8B is a graph showing fat volume in control (CON) and vibrated (LMMS) mice. Total fat volume was decreased by 28.5% (p=0.030) after 12 weeks of daily treatment with LMMS.

FIG. 8C graph showing epididymal fat pad weight at sacrifice in the control (CON) and vibrated (LMMS) mice of FIG. 8A.

FIG. 9A is an image of high resolution scans of the proximal tibia (600 mm region, 300 mm below growth plate) done ex vivo demonstrate the anabolic effect of low magnitude, high frequency mechanical stimulation to bone.

FIG. 9B is a graph showing bone volume fraction in control (CON) and LMMS treated mice. LMMS animals showed significant enhancements in bone volume fraction.

FIG. 9C is a graph showing trabecular number in control (CON) and LMMS treated mice. LMMS animals showed significant enhancements in trabecular number.

FIG. 10A and FIG. 10B are representative dot plots from flow cytometry experiments demonstrating the ability of LMMS to increase the number of cells expressing Stem Cell Antigen-1 (Sca-1). Cells in this experiment were double-labeled with Sca-1 (to identify MSCs, y-axis) and Preadipocyte factor (Pref-1, x-axis) to identify preadipocytes. Sca-1 only cells (highlighted, upper left) represent the population of uncommitted stem cells.

FIG. 10C is a graphical representation of the data in FIG. 10A and FIG. 10B. The actual increase in stem cell number was calculated as % positive cells/total number of bone marrow cells. RD denotes an age-matched control group of animals fed a regular diet, FD denotes fat diet fed animals. Regardless of diet, LMMS treatment increases the number of Sca-1 positively labeled cells.

FIG. 11A is a graph showing the percentage of GFP positive cells harvested from various tissues in control (CON) or vibrated (LMMS) mice. LMMS treatment was administered for 6 weeks. (N=8). (B) The reduced ratio of adipocytes shown relative to bone marrow GFP expression in LMMS indicates reduced commitment to fat. Ratio of adipocytes to blood is shown as a constant engraftment control.

### DETAILED DESCRIPTION

We further describe below the present methods for applying physical stimuli to subjects. These methods can be applied in, and are expected to benefit subjects in, a great variety of circumstances that arise in the context of, for example, traumatic injury (including that induced by surgical procedures), wound healing (of the skin and other tissues), cancer therapies (e.g., chemotherapy or radiation therapy), tissue transplantation (e.g., bone marrow transplantation), and aging. More generally, the present methods apply where patients would benefit from an increase in the number of cells (e.g., stem cells) within a given tissue and, ex vivo, where it is desirable to increase the proliferation of cells (e.g., stem cells) for scientific study, inclusion in devices bearing cells (e.g., polymer or hydrogel-based implants), and administration to patients.

The methods are based, inter alia, on our findings that even brief exposure to high frequency, low magnitude physical signals (e.g., mechanical signals), inducing loads below those that typically arise even during walking, have marked effects on the proliferation and differentiation of cells, including stem cells such as mesenchymal stem cells. The marked response to low and brief signals in the phenotype of a growing animal suggests the presence of an inherent physiologic process that has been previously unrecognized.

More specifically, we have found that non-invasive mechanical signals can markedly elevate the total number of stem cells in the marrow, and can bias their differentiation towards osteoblastogenesis and away from adipogenesis, resulting in both an increase in bone density and less visceral fat. A pilot trial on young osteopenic women suggests that the therapeutic potential of low magnitude mechanical signals can be translated to the clinic, with an enhancement of bone and muscle mass, and a concomitant suppression of visceral fat formation.

Described herein are methods and materials for the use of low magnitude mechanical signals (LMMS), of a specific frequency, amplitude and duration, that can be used to enhance the viability and/or number of stem cells (e.g., in cell culture or in vivo), as well as direct their path of differentiation. The methods can be used to accelerate and augment the process of tissue repair and regeneration, help alleviate the complications of treatments (e.g., radio- and chemotherapy) which compromise stem cell viability, enhance the incorporation of tissue grafts, including allografts, xenografts and autografts, and stem the deleterious effects of aging, in terms of retaining the population and activity of critical stem cell populations.

Stem Cells

The methods of the invention can be used enhance or increase proliferation (as assessed by, e.g., the rate of cell division), of a cell and/or population of cells in culture. The cultured population may or may not be purified (i.e., mixed cell types may be present, as may cells at various stages of differentiation). Numerous cell types are encompassed by the methods of the invention, including adult stem cells (regardless of their tissue source), embryonic stem cells, stem cells obtained from, for example, the umbilical cord or umbilical cord blood, primary cell cultures and established cell lines. Useful cell types can include any form of stem cell. Generally, stem cells are undifferentiated cells that have the ability both to go through numerous cycles of cell-division while maintaining an undifferentiated state and, under appropriate stimuli, to give rise to more specialized cells. In addition, the present methods can be applied to stem cells that have at least partially differentiated (i.e., cells that express markers found in precursor and mature or terminally differentiated cells).

Adult stem cells have been identified in many types of adult tissues, including bone marrow, blood, skin, the gastrointestinal tract, dental pulp, the retina of the eye, skeletal muscle, liver, pancreas, and brain. Bone marrow is an especially rich source of stem cells and includes hematopoietic stem cells, which can give rise to blood cells, endothelial stem cells, which can form the vascular system (arteries and veins) and mesenchymal stem cells. Mesenchymal stem cells, also referred to as MSCs, marrow stromal cells, multipotent stromal cells, are multipotent stem cells that can differentiate into a variety of cell types, including osteoblasts, chondrocytes, myocytes, adipocytes, and beta-pancreatic islet cells.

The methods of the invention can also be used to enhance or increase the proliferation of cultured cell lines, including but, not limited to embryonic stem cell lines, for example, the human embryonic stem cell line NCCIT; the mouse embryonic stem cell line R1/E; mouse hematopoeitic stem cell line EML Cell Line, Clone 1. Such cell lines can be obtained from commercial sources or can be those generated by the skilled artisan from tissue samples or explants using methods known in the art. The origins of any given cell line can be analyzed using cell surface markers, for example, Sca-1 or Pref-1, or molecular analysis of gene expression profiles or functional assays.

The methods described here can be carried out by providing, to the subject, a low-magnitude and high-frequency physical signal, such as a mechanical signal. The physical signal can be administered other than by a mechanical force (e.g., an ultrasound signal that generates the same displacement can be applied to the subject), and the signal, regardless of its source, can be supplied (or applied or administered) on a periodic basis and for a time sufficient to maintain, improve, or inhibit a worsening of a population of cells (e.g., the proliferation of MSCs in culture).

Low-Magnitude High-Frequency Mechanical Signals

The treatments disclosed herein are unique, non-pharmacological interventions for a number of diseases and conditions, including obesity (e.g., diet-induced obesity) and diabetes. They can, however, also be applied in a prophylactic or preventative manner in order to reduce the risk that a patient will develop one of the diseases or conditions described herein; to reduce the severity of that disease or condition, should it develop; or to delay the onset or progression of the disease or condition. For example, the present methods can be used to treat patients who are of a recommended weight or who are somewhat overweight but are not considered clinically obese. Similarly, the present methods can be used to treat patients who are considered to be at risk for developing diabetes or who are expected to experience a transplant or traumatic injury (e.g., an incision incurred in the course of a surgical procedure).

The physical stimuli delivered to a subject (e.g., a human) can be, for example, vibration(s), magnetic field(s), and ultrasound. The stimuli can be generated with appropriate means known in the art. For example, vibrations can be generated by transducers (e.g., actuators, e.g., electromagnetic actuators), magnetic field can be generated with Helmholtz coils, and ultrasound can be generated with piezoelectric transducers.

The physical stimuli, if introduced as mechanical signals (e.g., vibrations), can have a magnitude of at least or about 0.01-10.0 g. As demonstrated in the Examples below, signals of low magnitude are effective. Accordingly, the methods described here can be carried out by applying at least or about 0.1-1.0 g (e.g., 0.2-0.5 g, inclusive (e.g., about 0.2 g, 0.25 g, 0.3 g, 0.35 g, 0.4 g, 0.45 g, or 0.50 g)) to the subject. The frequency of the mechanical signal can be at least or about 5-1,000 Hz (e.g., 15 or 20-200 Hz, inclusive (e.g., 30-90 Hz (e.g., 30, 35, 40, 45, 50, or 55 Hz)). For example, the frequency of the mechanical signal can be about 5-100 Hz, inclusive (e.g., about 40-90 Hz (e.g., 50, 60, 70, 80, or 90 Hz) or 20-50 Hz (e.g., about 20, 25, 30, 35 or 40 Hz), a combination of frequencies (e.g., a “chirp” signal from 20-50 Hz), as well as a pulse-burst of mechanical information (e.g., a 0.5 s burst of 40 Hz, 0.3 g vibration given at least or about every 1 second during the treatment period). The mechanical signals can be provided on a periodic basis (e.g., weekly or daily). The physical signals can last at least or about 2-60 minutes, inclusive (e.g., 2, 5, 10, 15, 20, 30, 45, or 60 minutes).

Providing low-magnitude, high-frequency mechanical signals can be done by placing the subject on a device with a vibrating platform. An example of a device that can be used is the JUVENT 1000 (by Juvent, Inc., Somerset, N.J.) (see also U.S. Pat. No. 5,273,028). The source of the mechanical signal (e.g., a platform with a transducer, e.g., an actuator, and source of an input signal, e.g., electrical signal) can be variously housed or situated (e.g., under or within a chair, bed, exercise equipment, mat (e.g., a mat used to exercise (e.g., a yoga mat)), hand-held or portable device, a standing frame or the like). The source of the mechanical signal (e.g., a platform with a transducer, e.g., an actuator and a source of an input signal, e.g., electrical signal) can also be within or beneath a floor or other area where people tend to stand (e.g., a floor in front of a sink, stove, window, cashier's desk, or art installation or on a platform for public transportation) or sit (e.g., a seat in a vehicle (e.g., a car, train, bus, or plane) or wheelchair). The signal can also be introduced through oscillatory acceleration in the absence of weightbearing (e.g., oscillation of a limb), using the same frequencies and accelerations as described above.

Electromagnetic field signals can be generated via Helmholtz coils, in the same frequency range as described above, and with in the intensity range of 0.1 to 1000 micro-Volts per centimeter squared. Ultrasound signals can be generated via piezoelectric transducers, with a carrier wave in the frequency range described herein, and within the intensity range of 0.5 to 500 milli-Watts per centimeter squared. Ultrasound can also be used to generate vibrations described herein.

The transmissibility (or translation) of signals through the body is high, therefore, signals originating at the source, e.g., a platform with a transducer and a source of, e.g., electrical, signal, can reach various parts of the body. For example, if the subject stands on the source of the physical signal, e.g., the platform described herein, the signal can be transmitted through the subject's feet and into upper parts of the body, e.g., abdomen, shoulders etc.

As described in the Examples below, high frequency, low magnitude mechanical signals were delivered to mice via whole body vibration. When considering the potential to translate this to the clinic, it is important to note that associations persist between vibration and adverse health conditions, including low-back pain, circulatory disorders and neurovestibular dysfunction (Magnusson et al., Spine 21:710-17, 1996), leading to International Safety Organization advisories to limit human exposure to these mechanical signals (International Standards Organization. Evaluation of Human Exposure to Whole-Body Vibration. ISO 2631/1. 1985. Geneva). At the frequency (90 Hz) and amplitude used in the studies described herein (0.4 g peak-to-peak), the exposure would be considered safe for over four hours each day.

The physical signals can be delivered in a variety of ways, including by mechanical means by way of Whole Body Vibration through a ground-based vibrating platform or weight-bearing support of any type. In the case of cells in culture, the culture dish can be placed directly on the platform. Optionally, the platform is incorporated within a cell culture incubator or fermentor so that the signals can be delivered to the cells in order to maintain the temperature and pH of the cell culture medium. For a whole organism, the platform can contacts the subject directly (e.g., through bare feet) or indirectly (e.g., through padding, shoes, or clothing). The platform can essentially stand alone, and the subject can come in contact with it as they would with a bathroom scale (i.e., by simply stepping and standing on an upper surface). The subject can also be positioned on the platform in a variety of other ways. For example, the subject can sit, kneel, or lie on the platform. The platform may bear all of the patient's weight, and the signal can be directed in one or several directions. For example, a patient can stand on a platform vibrating vertically so that the signal is applied in parallel to the long axis of, for example, the patient's tibia, fibula, and femur. In other configurations, a patient can lie down on a platform vibrating vertically or horizontally. A platform that oscillates in several distinct directions could apply the signal multi-axially, e.g, in a non-longitudinal manner around two or more axes. Devices can also deliver the signal focally, using local vibration modalities (e.g., to the subject's abdomen, thighs, or back), as well as be incorporated into other devices, such as exercise devices. The physical signals can also be delivered by the use of acceleration, allowing a limb, for example, to oscillate back and forth without the need for direct load application, thus simplifying the constraints of local application modalities (e.g., reducing the build-up of fat in limb musculature following joint replacement).

Our studies have demonstrated that six weeks of LMMS in C57BL/6J mice can increase the overall marrow-based stem cell population by 37% and the number of MSCs by 46%. Concomitant with the increase in stem cell number, the differentiation postential of MSCs in the bone marrow was biased toward osteoblastic and against adipogenic differentiation, as reflected by upregulation of the transcription factor Runx2 by 72% and downregulation of PPARγ by 27%. The phenotypic impact of LMMS on MSC lineage determination was evident at 14 weeks, where visceral adipose tissue formation was suppressed by 28%.

Accordingly, the present methods employ mechanical signals as a non-invasive means to influence stem cell (e.g., mesenchymal stem cell) or precursor cell proliferation and fate (differentiation). In some instances, that influence will promote bone formation while suppressing the fat phenotype.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1

### Materials and Methods

Animal Model to Prevent Diet Induced Obesity (DIO). All animal procedures were reviewed and approved by the Stony Brook University animal care and use committee. The overall experimental design consisted of two similar protocols, differing in the duration of treatment to assess mechanistic responses of cells to LMMS (6 w of LMMS compared to control, n=8 per group) or to characterize the phenotypic effects (14 w of LMMS compared to control). Two models of DIO were employed: 1. to examine the ability of LMMS to prevent obesity, a “Fat Diet” condition (n=12 each, LMMS and CON) was evaluated where LMMS and DIO were initiated simultaneously, and 2. to examine the ability of LMMS to reverse obesity, an “Obese” condition (n=8 each, LMMS and CON) was established, whereby LMMS treatment commenced 3 weeks after the induction of DIO, and compared to sham controls.

Mechanical enhancement of stem cell proliferation and differentiation in DIO. Beginning at 7 w of age, C57BL/6J male mice were given free access to a high fat diet (45% kcal fat, # 58V8, Research Diet, Richmond, Ind.). The mice were randomized into two groups defined as LMMS (5d/w of 15 min/d of a 90 Hz, 0.2 g mechanical signal, where 1.0 g is earth's gravitational field, or 9.8 m/s2), and placebo sham controls (CON). The LMMS protocol 13 provides low magnitude, high frequency mechanical signals by a vertically oscillating platform, 14 and generates strain levels in bone tissue of less than five microstrain, several orders of magnitude below peak strains generated during strenuous activity. Food consumption was monitored by weekly weighing of food.

Status of MSC pool by flow cytometry. Cellular and molecular changes in the bone marrow resulting from 6 w LMMS (n=8 animals per group, CON or LMMS) were determined at sacrifice from bone marrow harvested from the right tibia and femur (animals at 13 w of age). Red blood cells in the bone marrow aspirate were removed by room temperature incubation with Pharmlyse (BD Bioscience) for 15 mins. Single cell suspensions were prepared in 1% sodium azide in PBS, stained with the appropriate primary and (when indicated) secondary antibodies, and fixed at a final concentration of 1% formalin in PBS. Phycoerythrin (PE) conjugated rat anti-mouse Sca-1 antibody and isotype control were purchased from BD Pharmingen and used at 1:100. Rabbit anti-mouse Pref-1 antibody and FITC conjugated secondary antibody were purchased from Abeam (Cambridge, Mass.) and used at 1:100 dilutions. Flow cytometry data was collected using a Becton Dickinson FACScaliber flow cytometer (San Jose, Calif.).

RNA extraction and real-time RT-PCR. At sacrifice, the left tibia and femur were removed and marrow flushed into an RNAlater solution (Ambion, Foster City, Calif.). Total RNA was harvested from the bone marrow using a modified TRIspin protocol. Briefly, TRIzol reagent (Life Technologies, Gaithersburg, Md.) was added to the total bone marrow cell suspension and the solution homogenized. Phases were separated with chloroform under centrifugation. RNA was precipitated via ethanol addition and applied directly to an RNeasy Total RNA isolation kit (Qiagen, Valencia, Calif.). DNA contamination was removed on column with RNase free DNase. Total RNA was quantified on a Nanodrop spectrophotometer and RNA integrity monitored by agarose electrophoresis. Expression levels of candidate genes was quantified using a real-time RT-PCR cycler (Lightcycler, Roche, Ind.) relative to the expression levels of samples spiked with exogenous cDNA. 15 A “one-step” kit (Qiagen) was used to perform both the reverse transcription and amplification steps in one reaction tube.

qRT-PCR with Content Defined 96 Gene Arrays. PCR arrays were obtained from Bar Harbor Biotech (Bar Harbor, Me.), with each well of a 96 well PCR plate containing gene specific primer pairs. The complete gene list for the osteoporosis array can be found at www.bhbio.com, and include genes that contribute to bone mineral density through bone resorption and formation, genes that have been linked to osteoporosis, as well as biomarkers and gene targets associated with therapeutic treatment of bone loss. cDNA samples were reversed transcribed (Message Sensor RT Kit, Ambion, Foster City, Calif.) from total RNA harvested from bone marrow cells and used as the template for each individual animal. Data were generated using an Applied Biosystems 7900HT real-time PCR machine, and analyzed by Bar Harbor Biotech.

Body habitus established by in vivo microcomputed tomography (μCT). Phenotypic effects of DIO, for both the “prevention” and “reversal” of obesity test conditions were defined after 12 and 14 w of LMMS. At 12 w, in vivo μCT scans were used to establish fat, lean, and bone volume of the torso (VivaCT 40, Scanco Medical, Bassersdorf, Switzerland). Scan data was collected at an isotropic voxel size of 76 μm (45 kV, 133 μA, 300-ms integration time), and analyzed from the base of the skull to the distal tibia for each animal. Threshold parameters were defined during analysis to segregate and quantify fat and bone volumes. Lean volume was defined as animal volume that is neither fat nor bone, and includes muscle and organ compartments.

Bone phenotype established by ex vivo microcomputed tomography. Trabecular bone morphology of the proximal region of the left tibia of each mouse was established by μCT at 12 μm resolution (μCT 40, Scanco Medical, Bassersdorf, Switzerland). The metaphyseal region spanned 600 μm, beginning 300 μm distal to the growth plate. Bone volume fraction (BV/TV), connectivity density (Conn.D), trabecular number (Tb.N), trabecular thickness (Tb.Th), trabecular separation (Tb.Sp), and the structural model index (SMI) were determined.

Serum and tissue biochemistry. Blood collection was performed after overnight fast by cardiac puncture with the animal under deep anesthesia. Serum was harvested by centrifugation (14,000 rpm, 15 min, 4° C.). Mice were euthanized by cervical dislocation, and the different tissues (i.e., epididymal fat pad and subcutaneous fat pads from the lower torso, liver, and heart) were excised, weighed, frozen in liquid nitrogen, and stored at −80° C. Total lipids from white adipose tissue (epididymal fat pad) and liver were extracted and purified based on a chloroform-methanol extraction. Total triglycerides (TG) and non-esterified free fatty acids (NEFA) were measured on serum (n=10 per group) and lipid extracts from adipose tissue (n=5 or 6 per group) and liver (n=10 per group) using enzymatic colorimetric kits (TG Kit from Sigma, Saint Louis, Mo.; and NEFA C from Wako Chemicals, Richmond, Va.). ELISA assays were utilized to determine serum concentrations of leptin, adiponectin, resistin (all from Millipore, Chicago, Ill.), osteopontin (R&D Systems, Minneapolis, Minn.), and osteocalcin (Biomedical Technologies Inc, Stoughton, Mass.), using a sample size of n=10 per group.

Human pilot trial to examine inverse relationship of adipogenesis and osteoblastogenesis. A trial designed and conducted to evaluate if 12 months of LMMS could promote bone density in the spine and hip of women with low bone density was evaluated retrospectively to examine changes in visceral fat volume. All procedures were reviewed and approved by the Childrens Hospital of Los Angeles Committee on Research in Human Subjects.

Forty-eight healthy young women (aged 15-20 years) were randomly assigned into either LMMS or CON groups (n=24 in each group). The LMMS group underwent brief (10 min requested), daily treatment with LMMS (30 Hz signal @0.3 g) for one year. Computed tomographic scans (CT) were performed at baseline and one year, with the same scanner (model CT-T 9800, General Electric Co., Milwaukee, Wis.), the same reference phantom for simultaneous calibration, and specially designed software for fat and muscle measurements. Identification of the abdominal site to be scanned was performed with a lateral scout view, followed by a cross-sectional image obtained from the midportion of the third lumbar vertebrae at 80 kVp, 70 milliamperes, and 2S.

Cancellous bone of the 1st, 2nd and 3rd lumbar vertebrae was established as measures of the tissue density of bone in milligrams per cubic centimeter (mg/cm3). Area of visceral fat (cm2) was defined at the midportion of the third lumbar vertebrae as the intra-abdominal adipose tissue surrounded by the rectus abdominus muscles, the external oblique muscles, the quadratus lumborum, the psoas muscles and the lumbar spine at the midportions of the third lumbar vertebrae, and consisted mainly of perirenal, pararenal, retroperitoneal and mesenteric fat. The average area of paraspinous musculature (cm2) was defined as the sums of the area of the erector spinae muscles, psoas major muscles and quadratus lumborum muscles at the midportion of the third lumbar vertebrae. 18 All analyses of bone density, and muscle and fat area were performed by an operator blinded as to subject enrollment.

Statistical analyses. All data are shown as mean±standard deviation, unless noted. To determine significant differences between LMMS and CON groups, two tailed t-tests (significance value set at 5%) were used throughout. Animal outliers were determined based on animal weight at baseline (before the start of any treatment) as animals falling outside of two standard deviations from the total population, or in each respective group at the end of 6 or 14 weeks LMMS (or sham CON) by failure of the Weisberg one-tailed t-test (alpha=0.01), regarded as an objective tool for showing consistency within small data sets. 19 No outliers were identified in the 6 w CON and LMMS groups. Two outliers per group (CON and LMMS) were identified in the Fat Diet model (14 w LMMS study) and removed. Data from these animals were not included in any analyses, resulting in a sample size of n=10 per group for all data, unless otherwise noted. No outliers were identified in the 14 w Obese model (n=8). Data presented from the human trial are based on the intent to treat data set (all subjects included in the evaluation). Changes in visceral fat volume were compared between LMMS and CON subjects using a one tailed t-test.

### Example 2

### Bone Marrow Stem Cell Population is Promoted by LMMS

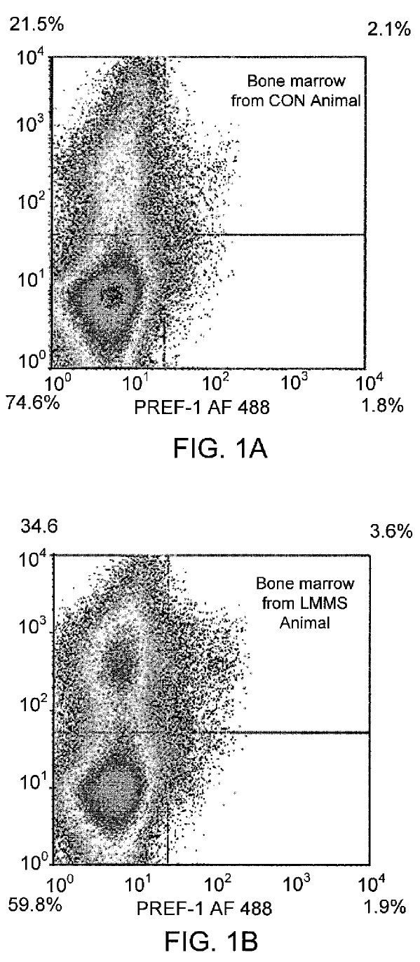

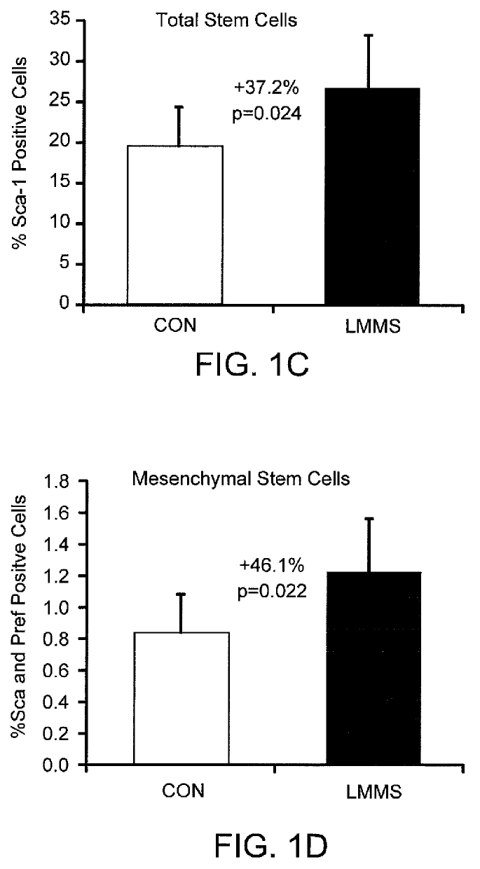

Flow cytometric measurements using antibodies against Stem Cell Antigen-1 (Sca-1) indicated that in animals in the “prevention” DIO group, 6 w of LMMS treatment significantly increased the overall stem cell population relative to controls, as defined by cells expressing Sca-1. Analysis focused on the primitive population of cells with low forward (FSC) and side scatter (SSC), indicating the highest Sca-1 staining for all cell populations. Cells in this region demonstrated a 37.2% (p=0.028) increase in LMMS stem cell numbers relative to sham CON animals. Mesenchymal stem cells as represented by cells positive for Sca-1 and Preadipocyte Factor-1 (Pref-1), 1 represented a much smaller percentage of the total cells. Identified in this manner, in addition to the increase in the overall stem cell component, LMMS treated animals had a 46.1% (p=0.022) increase in mesenchymal stem cells relative to CON (FIG. 1).

### Example 3

### LMMS Biases Marrow Environment and Lineage Commitment

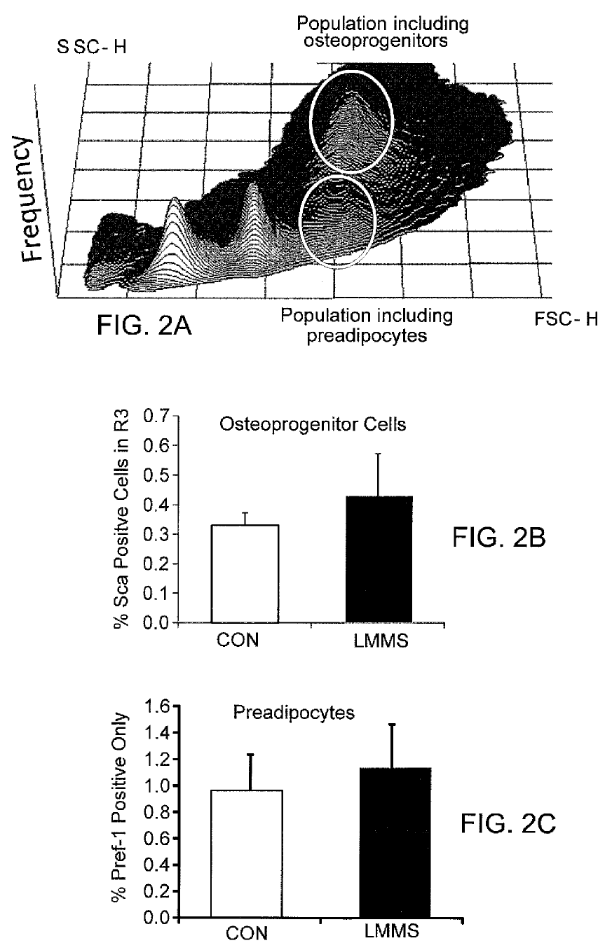

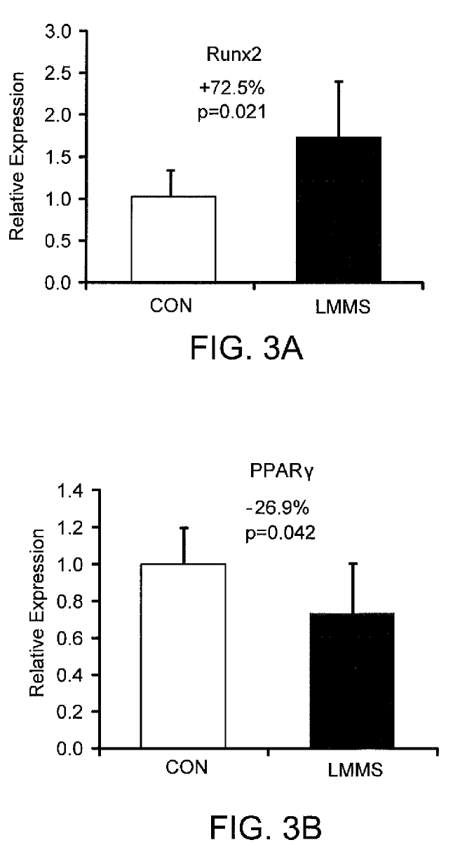

After six weeks, cells expressing only the Pref-1 label, considered committed preadipocytes, were elevated by 18.5% (p=0.25) in LMMS treated animals relative to CON (FIG. 2). Osteoprogenitor cells in the bone marrow population, identified as Sca-1 positive with high FSC and SSC, 20 were 29.9% greater (p=0.23) greater when subject to LMMS. This trend indicating that differentiation in the marrow space of LMMS animals had shifted towards osteogenesis was confirmed by gene expression data, which demonstrated that transcription of Runx2 in total bone marrow isolated from LMMS animals was upregulated 72.5% (p=0.021) relative to CON. In these same LMMS animals, expression of PPARγ was downregulated by 26.9% (p=0.042) relative to CON (FIG. 3).

Gene expression data on bone marrow samples were also tested on a 96 gene “osteoporosis” array, which included genes that contribute to bone mineral density through bone resorption and formation, and genes that have been linked to osteoporosis through association studies. Samples for both CON and LMMS groups expressed 83 of the 94 genes present on the array. qRT-PCR arrays reported decreases in genes such as Pon1 (paraoxonase-1), is known to be associated with high density lipoproteins (−137%, p=0.263), and sclerostin (−258%, p=0.042), which antagonizes bone formation by acting on Wnt signaling. 21 Genes such as estrogen related receptor (Esrra; +107%, p=0.018) and Pomc-1 (pro-opiomelanocortin, +68%, p=0.055) were up-regulated by LMMS.

### Example 4

### LMMS Enhancement of Bone Quantity and Quality

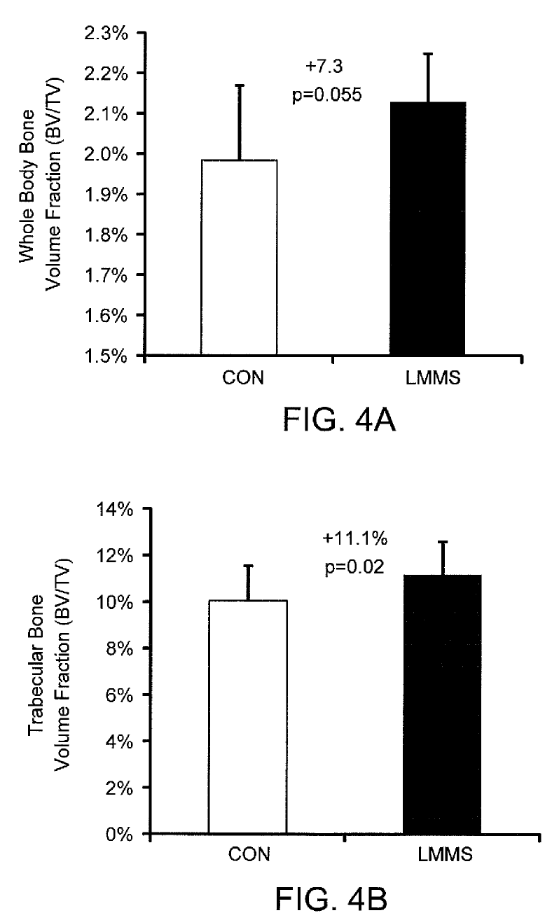

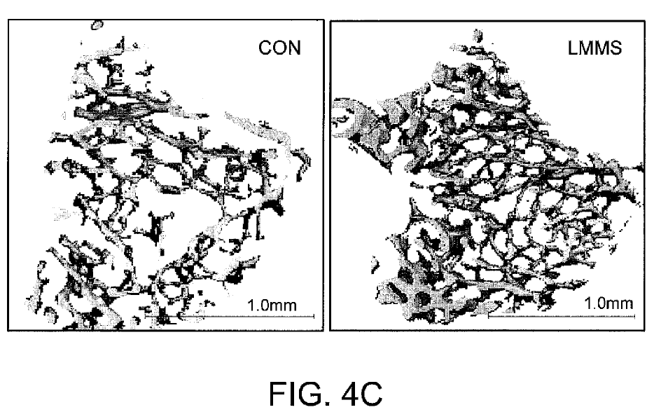

The ability of LMMS induced changes in proliferation and differentiation of MSCs to elicit phenotypic changes in the skeleton was first measured at 12 w by in vivo μCT scanning of the whole mouse (neck to distal tibia). Animals subject to LMMS showed a 7.3% (p=0.055) increase in bone volume fraction of the axial and appendicular skeleton (BV/TV) over sham CON. Post-sacrifice, 12 μm resolution μCT scans of the isolated proximal tibia of the LMMS animals showed 11.1% (p=0.024) greater bone volume fraction than CON (FIG. 4). The micro architectural properties were also enhanced in LMMS as compared to CON, as evidenced by 23.7% greater connectivity density (p=0.037), 10.4% higher trabecular number (p=0.022), 11.1% smaller separation of trabeculae (p=0.017) and a 4.9% lower structural model index (SMI, p=0.021; Table 1).

### Example 5

### Prevention of Obesity by LMMS

At 12 w, neither body mass gains nor the average weekly food intake differed significantly between the LMMS or CON groups (Table 2). At this point (19 wks of age), CON weighed 32.9 g±4.2 g, while LMMS mice were 6.8% lighter at 30.7 g±2.1 g (p=0.15). CON were 15.0% heavier than mice of the same strain, gender and age that were fed a regular chow diet, 13 and increase in body mass due to high fat feeding was comparable to previously reported values. 22 Adipose volume from the abdominal region (defined as the area encompassing the lumbar spine) was segregated as either subcutaneous or visceral adipose tissue (SAT or VAT, respectively). LMMS animals had 28.5% (p=0.021) less VAT by volume, and 19.0% (p=0.016) less SAT by calculated volume. Weights of epididymal fat pads harvested at sacrifice (14 w) correlated strongly with fat volume data obtained by CT. The epididymal fat pad weight was 24.5% (p=0.032) less in LMMS than CON, while the subcutaneous fat pad at the lower back region was 26.1% (p=0.018) lower in LMMS (Table 2).

### Example 6

### LMMS Prevents Increased Biochemical Indices of Obesity

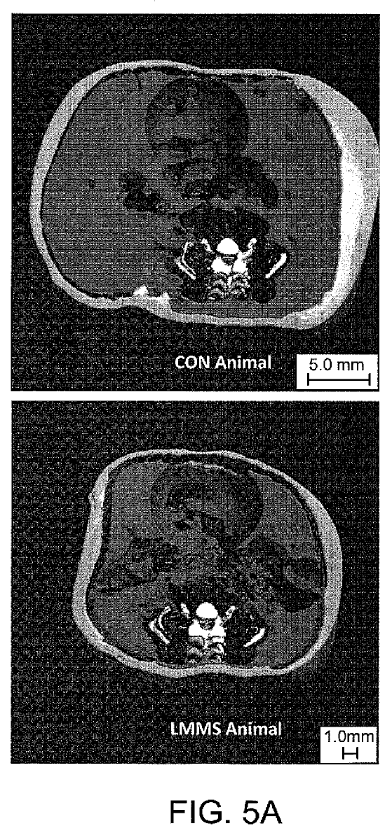

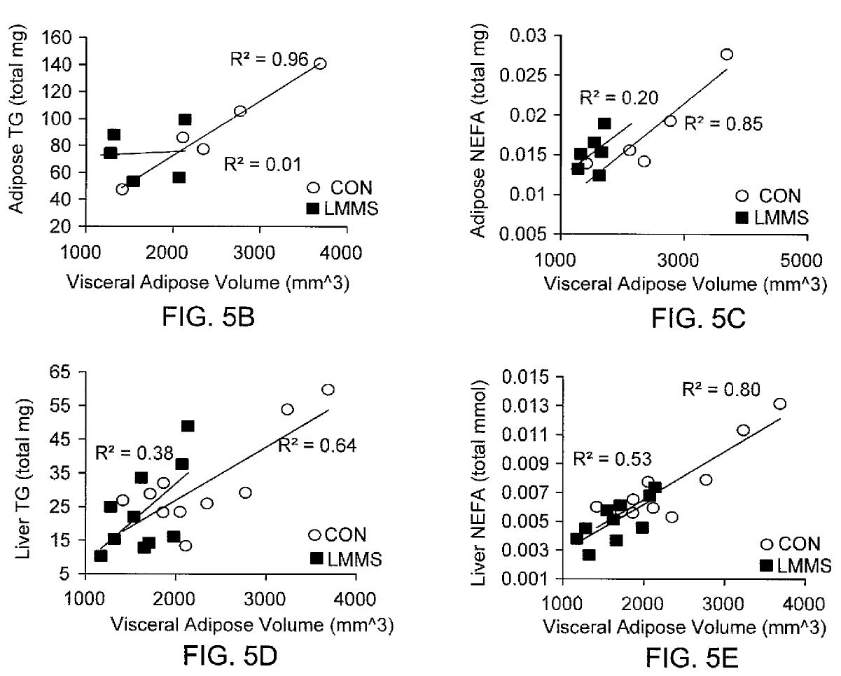

Triglycerides (TG) and non-esterified free fatty acids (NEFA) measured in plasma, epididymal adipose tissue, and liver were all lower in LMMS as compared to CON (Table 3). Liver TG levels decreased by 25.6% (p=0.19) in LMMS animals, paralleled by a 33.0% (p=0.022) decrease in NEFA levels. Linear regressions of adipose and liver TG and NEFA values to μCT visceral volume (VAT) demonstrated strong positive correlations for CON animals, with R2=0.96 (p=0.002) for adipose TG, R2=0.85 (p=0.027) for adipose NEFA, R2=0.64 (p=0.006) for liver TG and R2=0.80 (p=0.003) for liver NEFA (FIG. 5). LMMS resulted in weaker correlations between all TG and NEFA levels to increases in VAT.

At sacrifice, fasting serum levels of adipokines were lower in LMMS as compared to CON. Circulating levels of leptin were 35.3% (p=0.05) lower, adiponectin was 21.8% (p=0.009) lower, and resistin was 15.8% lower (p=0.26) than CON (Table 3). Circulating serum osteopontin (−7.5%, p=0.41) and osteocalcin (−14.6%, p=0.22) levels were not significantly affected by the mechanical signals.

### Example 7

### LMMS Fails to Reduce Existing Adiposity

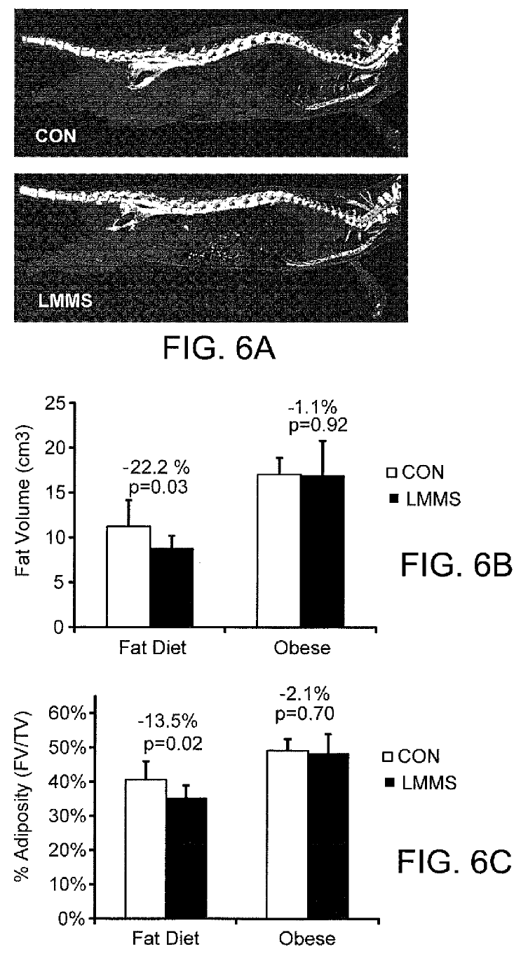

In the “reversal” model of obesity, 4 w old animals were started on a high fat diet for 3 w prior to beginning the LMMS protocol at 7 w of age. These “obese” animals were on average 3.7 grams heavier (p<0.001) than chow fed regular diet animals (baseline) at the start of the protocol. The early-adolescent obesity in these mice translated to to adulthood, such that by the end of the 12 w protocol, they weighed 21% more than the CON animals who begun the fat diet at 7 w of age (p<0.001). In stark contrast to the “prevention” animals, where LMMS realized a 22.2% (p=0.03) lower overall adipose volume relative to CON (distal tibia to the base of the skull), no differences were seen for fat (−1.1%, p=0.92), lean (+1.3%, p=0.85), or bone volume (−0.2%, p=0.94) between LMMS and sham control groups after 12 w of LMMS for these already obese mice (FIG. 6).

### Example 8

### LMMS Promotes Bone and Muscle and Suppresses Visceral Fat

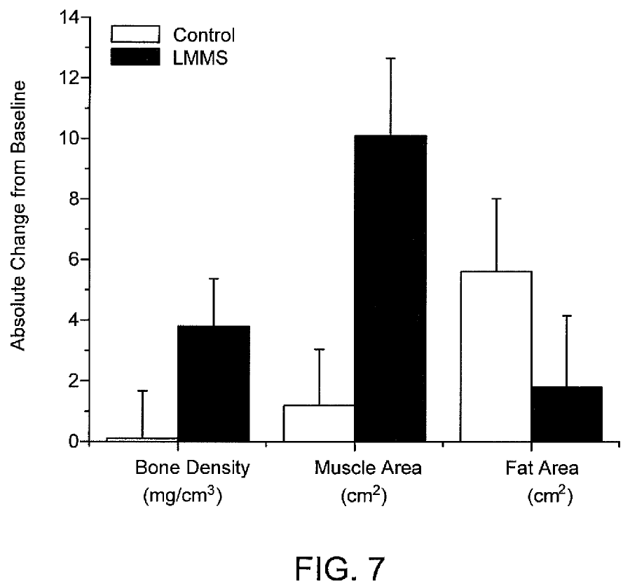

To determine whether the capacity of LMMS to suppress adiposity and increase osteogenesis in mice can translate to the human, young women with low bone density were subject to daily exposure to LMMS for 12 months. The study cohort ranged from 15-20 years old, and represented an osteopenic cohort. Detailed descriptions of this study population are provided elsewhere. 18 Over the course of one year, women (n=24) in the CON group had no significant change in cancellous bone density of the spine (0.1 mg/cm³±s.e. 1.5; FIG. 7), as compared to a 3.8 mg/cm³±1.6 increase in the spine of the LMMS treated cohort (p=0.06). Further, the average area of paraspinous muscle at the midportion of the third lumbar vertebrae, which failed to change in CON (1.2 cm²±1.9), was sharply elevated in the LMMS women (10.1 cm²±2.5; p=0.002). The area of visceral fat measured at the lumbrosacral region of CON subjects increased significantly from baseline by 5.6 cm²±2.4 (p=0.015). In contrast, the area of visceral fat measured in LMMS subjects increased by only 1.8 cm²±2.3, which was not significantly different from baseline (p=0.22). The 3.8 cm² difference in visceral fat area between groups showed a trend towards significance (p=0.13).

### Example 9

### LMMS Effects on Adipose Tissue Volume and Distribution

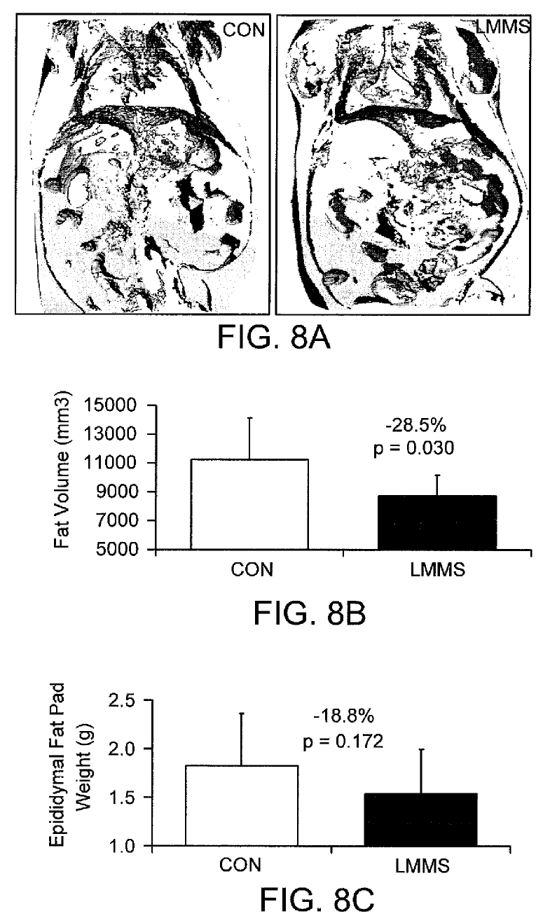

In a mouse model of dietary induced obesity, young male C57/B16 mice were fed a high fat diet where the fat content represented 45% of the calories. The LMMS stimulus (90 Hz, 0.2 g acceleration) was applied to the treatment group (n=12) for 15 min/d, 5 d/wk. A control group of animals fed the same diet but not treated with LMMS was maintained. After twelve weeks of treatment, the LMMS animals exhibited a statistically significant 28.5% reduction in total adipose volume when compared to the untreated controls, as measured by whole body vivaCT scanning. The whole body images were digitally filtered and segmented so that only fat tissue (excluding bone, organs, and muscle) would be measured. When the animals were sacrificed two weeks later, the epididymal fat pad was harvested from each animal and weighed. The decrease in fat volume based on image analysis was paralleled by a decrease of the weight of the actual epididymal fat pad harvested at sacrifice. (FIG. 8).

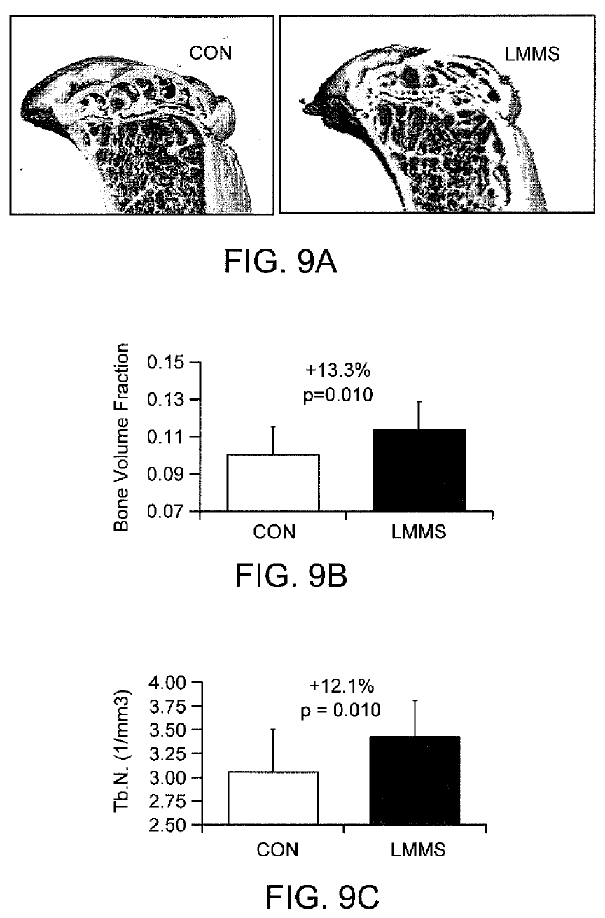

In parallel to measured decrease in fat weight and volume, these same animals exhibited an increase in their trabecular bone volume. In the proximal tibia, LMMS treated animals showed an increase in bone volume fraction of 13.3%. Microarchitectural parameters of connectivity density and trabecular number were also significantly increased, indicating better quality of bone (FIG. 9).

### Example 10

### LMMS Effects on Mesenchymal Stem cell Numbers

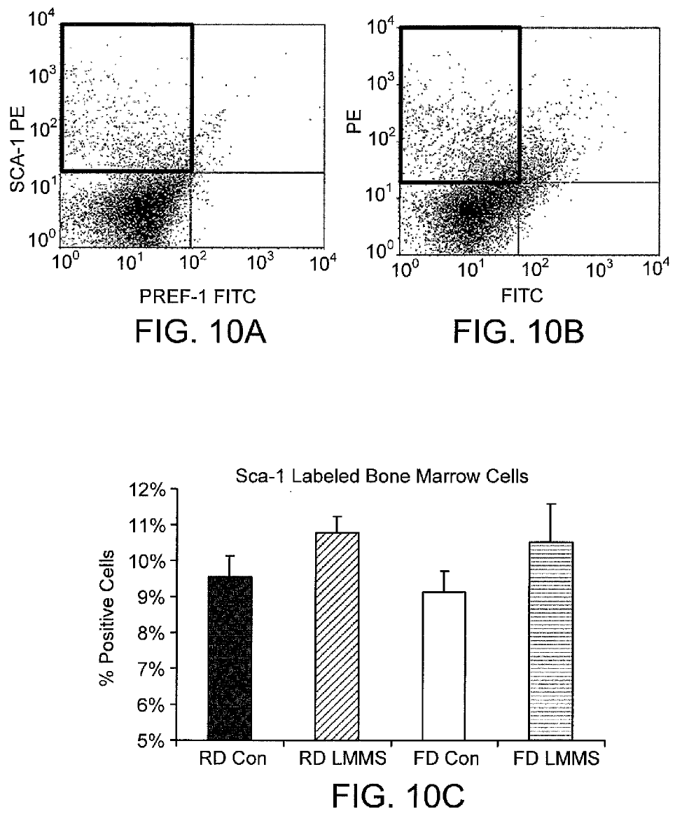

Using flow cytometry, mesenchymal stem cells can be identified out of a population of total bone marrow harvested cells by surface staining for Stem Cell Antigen-1 (Sca-1). Fluorescence conjugated anti-Sca-1 antibodies will bind only to cells expressing this surface antigen, including MSCs, allowing an accurate method to quantify stem cell number between different populations. With this method, it was demonstrated that 6 weeks of LMMS treatment applied via whole body vibration to a mouse can increase the number of MSC's by a statistically significant 19.9% (p=0.001). (FIG. 10)

### Example 11

### LMMS Effects on Stem Cell Proliferation in a Bone Marrow Transplant Model

To determine the ability of the LMMS signal to direct the differentiation pathway of stem cells, we utilized a bone marrow transplant model where GFP labeled bone marrow from a heterozygous animals was harvested and injected into sub-lethally irradiated wild-type mice. The GFP transplanted cells localize to the bone marrow cavity in the recipient mice, and repopulate the radiation damaged cells. With this model, it is possible to track the differentiation of stem cells as they retain their green fluorescence even after fully differentiating into a mature cell type. We subjected a population of bone marrow transplanted mice to 6 weeks of the LMMS treatment. At sacrifice, bone marrow, blood (after treatment to lyse the red blood cells), and adipocytes isolated by collagenase digestion from the epididymal fat pad were harvested and analysed by flow cytometry for GFP expression to track cell differentiation. Flow cytometry data utilized non-treated, age matched bone marrow transplant control animals as basal “normalization” controls.

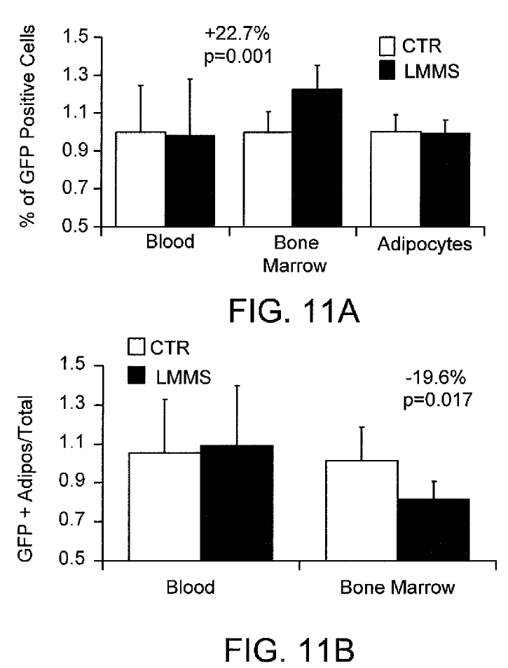

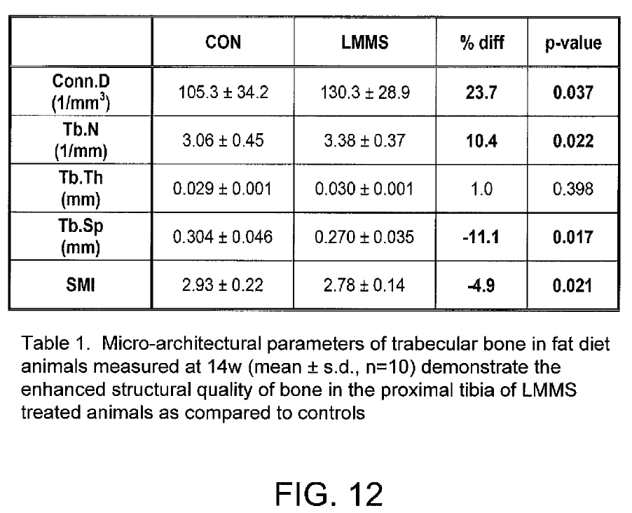

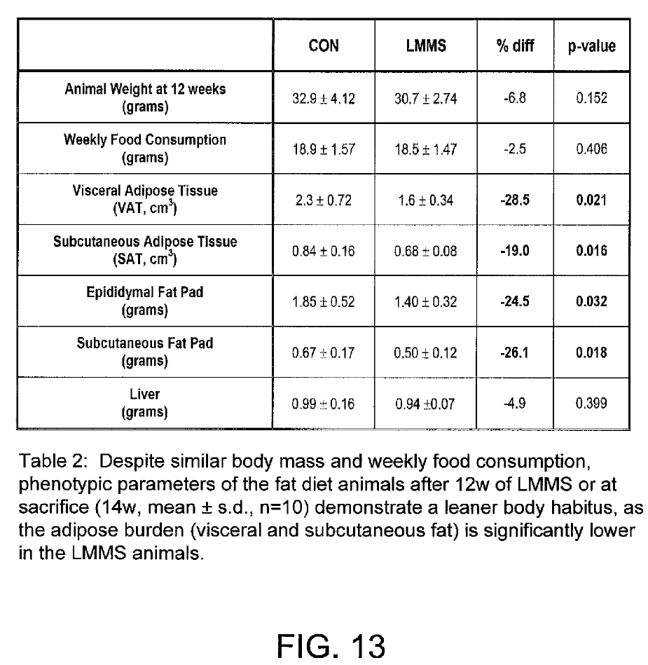

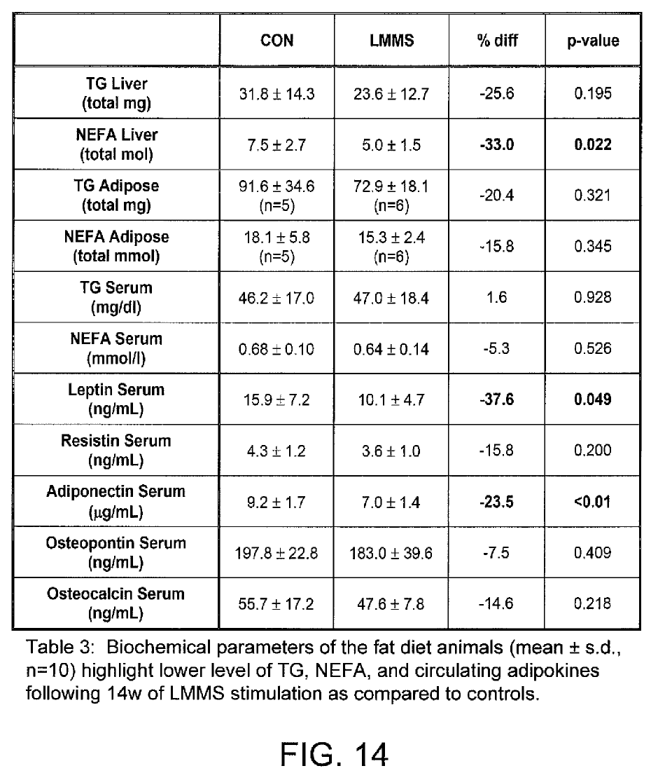

FIG. 11 summarizes data collected from the bone marrow transplant animal study. We confirm data presented in FIG. 3, that LMMS treatment increased the amount of GFP positive cells in the marrow compartment (+22.7%, p=0.001). In addition, normalized to the increased number of progenitor cells (MSCs), the number of GFP positive adipocytes was reduced by 19.6%, showing that fewer cells were differentiating into adipose tissue (FIG. 11.)

1. A method of increasing the proliferation of a cell, the method comprising administering to the cell a low magnitude, high frequency mechanical signal on a periodic basis and for a time sufficient to enhance or increase the proliferation of the cell to an extent greater than would be expected in the absence of the low magnitude, high frequency mechanical signal.

2. The method of claim 1, wherein the cell is a cell that has been placed in culture.

3. The method of claim 1, wherein the cell is a cell in vivo.

4. The method of claim 1, wherein the cell is a stem cell.

5.-8. (canceled)

9. The method of claim 1, wherein the cell has been modified to express an exogenous gene.

10. The method of claim 1, wherein the magnitude of the mechanical signal is about 0.01-10.0 g.

11. The method of claim 10, wherein the magnitude of the mechanical signal is about 0.2-0.5 g.

12. The method of claim 11, wherein the magnitude of the mechanical signal is about 0.3 g.

13. The method of claim 1, wherein the frequency of the mechanical signal is about 5-1000 Hz.

14. The method of claim 13, wherein the frequency of the mechanical signal is about 30-100 Hz.

15. The method of claim 14, wherein the frequency of the mechanical signal is about 90 Hz.

16. The method of claim 1, wherein the periodic basis is a daily or weekly basis.

17. The method of claim 1, wherein the time is about 2-200 minutes.

18. The method of claim 1, wherein providing the low magnitude, high frequency mechanical signal comprises placing the cell on an article comprising a vibrating platform that delivers the low magnitude, high frequency mechanical signal to the subject.

19.-21. (canceled)

22. The method of claim 1, further comprising the step of identifying a suitable subject to whom the signal would be administered.

23. (canceled)

24. A method of treating a patient, the method comprising administering to the patient a cell that has been treated according to the method of claim 1.

25. A method of treating a patient who has experienced a traumatic injury to a tissue or who has a tissue damaging disease other than osteopenia or sarcopenia, the method comprising administering to the patient a low magnitude, high frequency mechanical signal on a periodic basis and for a time sufficient to treat the injury or tissue damage.

26.-30. (canceled)

31. A method of treating a patient who is undergoing chemotherapy or radiation therapy, or who has received a bone marrow transplant, the method comprising administering to the patient a low magnitude, high frequency mechanical signal on a periodic basis and for a time sufficient to counteract a harmful side effect of the chemotherapy or radiation therapy on the patient's body or to improve the outcome of the bone marrow transplant.

32. The method of claim 31, wherein the side effect is dry or discolored skin, palmar-plantar syndrome, damage to the skin caused by radiation or extravasation of the chemotherapeutic, hair loss, intestinal irritation, mouth sores or ulcers, cell loss from the bone marrow or blood, liver damage, kidney damage, lung damage, or a neuropathy.

33. The method of claim 31, wherein the patient is a human patient.

34.-35. (canceled)

36. A method of preparing a tissue donor, the method comprising administering to the donor a low magnitude, high frequency mechanical signal on a periodic basis and for a time sufficient to increase the number of cells in the tissue to be harvested for transplantation.

37. The method of claim 36, wherein the cells are stem cells and/or the tissue is bone marrow.

38.-45. (canceled)
